(19) United States
(12) Plant Patent Application Publication
(10) Pub. No.: US 2004/0040063 P1
(43) Pub. Date: Feb. 26, 2004

(54) Kalanchoe plant named 'PELLINORE'

(75) Inventor: A. Smaal, (US)

Correspondence Address: WOOD, PHILLIPS, KATZ, CLARK & MORTIMER, Suite 3800, 500 West Madison Street, Chicago, IL, 60661-2511, US

(73) Assignee: A.B. BREEDING BV

(21) Appl. No.: 10/644,669

(22) Filed: Aug. 20, 2003

Related U.S. Application Data
(63) Continuation of application No. 09/993,456, filed on Nov. 19, 2001.

(30) Foreign Application Priority Data
Jan. 11, 2001 (CA) 01-2521

(51) Int. Cl. A01H 5/00

(52) U.S. Cl. PLT/339

(57) ABSTRACT

A new and distinct cultivar of Kalanchoe plant named Pellinore is provided. This new cultivar is characterized by its bright pink flowers, dark green foliage, compact growth habit and excellent basal branching.

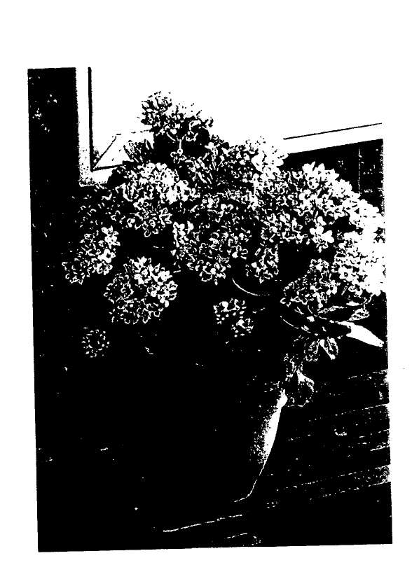

### BACKGROUND OF THE INVENTION

The present invention comprises a new and distinct Kalanchoe plant, botanically known as Kalanchoe blossfeldiana, and hereinafter referred to by the cultivar name ‘Pellinore’.

The new cultivar was developed by the inventor through a controlled breeding program during August 1996 in The Netherlands. The female (seed) parent of ‘Pellinore’ was the proprietary Kalanchoe blossfeldiana selection designated #92111-7 (not patented) characterized by its light pink flowers. ‘Pellinore’ was discovered and selected as a flowering plant within the progeny of the stated cross by the inventor in June 1997.

Asexual reproduction of the new cultivar by terminal cuttings has demonstrated that the characteristics of the new cultivar as herein described are firmly fixed and retained through successive generations of such asexual propagation.

### SUMMARY OF THE INVENTION

It was found that the cultivar of the present invention:

(a) exhibits bright pink flowers,

(b) forms dark green foliage,

(c) exhibits a good basal branching character and

(d) exhibits a compact upright growth habit.

The new cultivar of the present invention can be compared to ‘Light Jacqueline’ (U.S. Plant Pat. No. 10,268). In side by side comparisons with ‘Light Jacqueline’, it is found that the new cultivar is more compact, less floriferous and exhibits flowers of a brighter pink color. A detailed comparison can be found in Table A.

### BRIEF DESCRIPTION OF PHOTOGRAPH

The accompanying photograph shows as nearly true as it is reasonably possible to make the same in color illustrations of this type, typical flower and foliage characteristics of the new cultivar. The plants were grown in a greenhouse at West Chicago, Ill., U.S.A.

### DETAILED BOTANICAL DESCRIPTION

The ‘Pellinore’ cultivar has not been observed under all possible environmental conditions to date. Accordingly, it is possible that the phenotype may vary somewhat with variations in the environment, such as temperature, light intensity, and day length.

The chart used in the identification of colors described herein is the R.H.S. Colour Chart of The Royal Horticultural Society, London, England. The color values were determined on Jan. 4, 2001 between 9:00 and 9:30 am under natural daylight. The plants used for the following observations and measurements were produced from cuttings taken from stock plants and grown in West Chicago, Ill. in 10 cm pots for sixteen (16) weeks. Plants were grown in a double poly carbonate covered greenhouse under conditions comparable to those used in commercial practice while utilizing a soilless growth medium and maintaining temperatures of approximately 82° F. during the day and approximately 66° F. during the night. Plants were exposed to three weeks of long days followed by 9 weeks of short days. Growth regulators were applied as follows: B nine, 3000 ppm at days 17 and 28 and 1500 ppm day 42.

Classification:

Botanical.—Kalanchoe blossfeldiana, cultivar Pellinore.

Parantage:

Male(pollen)parent.—Proprietary Kalanchoe blossfeldiana selection #92111-7.

Female(seed)parent.—Proprietary Kalanchoe blossfeldiana selection #93074.

Propagation:

Type cutting.—Terminal tip.

Time to initiate roots.—Approximately 7-14 days with the shorter times generally being experienced in the summer and the longer times in the winter.

Time to develop roots.—Approximately 21 days.

Root description.—Fibrous, branching.

Plant Description:

Habit of growth.—Moderately vigorous with good basal branching. A mature plant, at 16 weeks after te planting of an unrooted cutting, commonly measures approximately 13.9 cm in height and approximately 16.1 cm in width.

Branching habit.—Typically forms one main stem 10 cm in length and 5 mm in diameter and 8 to 12 lateral branches approximately 8.3 cm in length and 4 mm in diameter.

Plant form.—Upright and mounded.

Foliage.—Form: Simple. Arrangement: Opposite. Shape: Ovate with obtuse apex, rounded base and crenate margin. Texture: Glabrous, succulent. Size of mature foliage: Approximately 7.8 cm in length and approximately 6.2 cm in width. Color of mature foliage: Upper surface between 137A and 139A; lower surface closest to 137B. Petiole length: Approximately 1 cm. Petiole color: 144B.

Flower description:

Flowering habit.—Freely flowering.

Natural flowering season.—Early January. Flowering time is dependent on temperature, light intensity and other growing conditions.

Flowering response time.—Approximately 11 weeks.

Inflorescence type.—Dichasial cymes.

Flowers Borne.—Above foliage.

Flower longevity.—Approximately 7 days.

Fragrance.—None.

Quantity of flowers.—Approximately 36 flowers on main stem and 23 flowers on each lateral branch.

Flower buds.—Shape: elliptical, becoming elongated as it matures. Size just before opening: Length is approximately 7 mm, width is approximately 4 mm. Color is 32C.

Flower.—Salverform. Corolla: Diameter is approximately 1.5 cm, depth is approximately 6 mm and aspect is cupped. Corolla tube is approximately 9 mm long, 3 mm in diameter.

Mature flower color.—Upper surface of petals is closest to 43C at opening, becoming 55B with midib blush of 54B at maturity and fading to 55D with midrib blush of 55B at senescence; lower surface of petals is closest to 55D.

Petals.—Number: Four. Shape: Obovate with cuspidate apex, rounded base and entire margin. Approximate size: 8 mm in length, 6 mm in width. Aspect: Slightly concave. Texture: Glabrous. Appearance: Somewhat iridescent.

Sepals.—Number: Four. Shape: Lanceolate with acuminate apex, entire margin. Size: 7 mm in length, 1 mm in width. Texture: Smooth. Color: Upper and lower surface: 144B.

Peduncle.—Approximately 2 cm in length and 4 mm in diameter.

Pedicel length.—Approximately 5 mm.

Reproductive organs.—Androecium: 8 stamens arranged in two groups of four—one group being shorter than the other. Anther shape is oblong and flat; length is approximately 0.5 mm. Pollen color is 3B. Gynoecium: Stigma shape is flat. Appearance is crystalline, colorless and transparent. Style length is approximately 1 mm; color is 154B. Ovary: 4 celled, 7 mm in length. Ovary color: 144B.

Seed development: Seed development has not been observed.

Disease resistance: Resistance to known Kalanchoe pathogens has not been observed.

TABLE A

| CHARACTERISTIC | ‘PELLINORE’ | ‘LIGHT JACQUELINE’ |
|---|---|---|
| PLANT HEIGHT | 13.9 cm | 21.8 cm |
| NUMBER OF | 10 | 12 |
| BRANCHES | | |
| COROLLA DIAMETER | 1.5 cm | 1.4 cm |
| PETAL COLOR-FULLY | 55B with midrib | Between 68A and 68B |
| OPENED-UPPER | blush of 54B | |
| SURFACE-MATURE | | |
| FLOWER | | |
| APPROXIMATE NUM- | 266 | 355 |
| BER OF FLOWERS | | |
| PER PLANT | | |
| LEAF LENGTH | 11.7 cm | 13.7 cm |
| LEAF WIDTH | 6.2 cm | 5.9 cm |

I claim:

1. A new and distinct cultivar of Kalanchoe plant named ‘Pellinore’ substantially as herein shown and described, which:
(a) exhibits bright pink flowers,
(b) forms dark green foliage,
(c) exhibits a good basal branching character and
(d) exhibits a compact upright growth habit.
